(19) United States
(12) Patent Application Publication
(10) Pub. No.: US 2024/0319050 A1
(43) Pub. Date: Sep. 26, 2024

(54) SYSTEMS AND DEVICES FOR IOT-ENABLED MONITORING OF METHANE EMISSIONS OF ONE OR MORE INDUSTRIAL FACILITIES

(71) Applicant: Schlumberger Technology Corporation, Sugar Land, TX (US)

(72) Inventors: Raphael Gadot, Houston, TX (US); Burc Abdullah Simsek, Sugar land, TX (US); Adam P. Huynh, Houston, TX (US)

(21) Appl. No.: 18/610,448

(22) Filed: Mar. 20, 2024

Related U.S. Application Data
(60) Provisional application No. 63/491,134, filed on Mar. 20, 2023.

(51) Int. Cl. G01N 1/22; G08C 17/02

(52) U.S. Cl. CPC G01N 1/2273; G08C 17/02

(57) ABSTRACT

The disclosure relates to sensors and other devices that are part of automated systems and methods for monitoring methane emissions at one or more industrial facilities.

### CROSS-REFERENCE TO RELATED APPLICATION(S)

The subject disclosure claims priority from U.S. Prov. Appl. No. 63/491,134, filed Mar. 20, 2023, entitled “SYSTEMS AND DEVICES FOR IOT-ENABLED MONITORING OF METHANE EMISSIONS OF ONE OR MORE INDUSTRIAL FACILITIES,” Attorney Docket No. IS22.1182-US-PSP, here incorporated by reference in its entirety.

### FIELD

The subject disclosure relates to systems and methods for monitoring methane emissions at industrial facilities, such as oil and gas facilities including well sites, compressor stations, and processing facilities.

### BACKGROUND

Methane emissions in the oil and gas industry are receiving intense scrutiny as it is believed that such methane emissions contribute to global warming and/or climate change. A large portion of the methane emissions in the oil and gas industry arise from a small number of major emission events henceforth referred to as super-emitters. Super-emitters occur from a variety of sites, and recent data suggest many emission events are intermittent.

Continuous monitoring using methane emissions detectors installed permanently at a site offers an effective way to identify, quantify, and mitigate intermittent methane emissions. However, installing methane emissions detectors across many diverse sites can be economically challenging.

### SUMMARY

In embodiments, an emissions detector for monitoring methane emissions at an industrial facility is provided that includes a gas sensor and an RF communication modem. The emissions detector is placed at a fixed location at or near the industrial facility. The gas sensor is configurable or configured to measure concentration of methane in atmospheric gas at or near the industrial facility. The RF communication modem is configurable or configured to communicate times-series sensor data based on such methane concentration measurements (i.e., high-frequency location-specific methane concentration data) directly to a radio access network over one or more RF communication links supported by the RF communication modem for delivery over a data communication network to a remote cloud computing environment.

In embodiments, the emissions detector can further include one or more atmospheric sensors that are configurable or configured to perform time-series measurements of atmospheric conditions (such as temperature, atmospheric pressure, and humidity) at or near the industrial facility. The RF communication modem can be configurable or configured to communicate times-series sensor data based on such atmospheric conditions (i.e., high-frequency location-specific atmospheric condition data) directly to the radio access network over one or more RF communication links supported by the RF communication modem for delivery over the data communication network to the remote cloud computing environment.

In embodiments, the emissions detector can also include one or more environmental sensors that are configurable or configured to perform time-series measurements of environmental conditions (such as wind speed, wind direction, and solar radiation) at or near the industrial facility. The RF communication modem can be configurable or configured to communicate times-series sensor data based on such measured environmental conditions (i.e., high-frequency location-specific environmental condition data) directly to the radio access network over one or more RF communication links supported by the RF communication modem for delivery over the data communication network to the remote cloud computing environment.

In embodiments, the RF communication modem and the radio access network can be configured to support wireless data communication of the time-series data over at least one direct RF communication link between the RF communication modem and the radio access network. In embodiments, the at least one direct RF communication link can implement at least one predefined wireless communication protocol having a range of ten kilometers or less (preferably the LTE-M protocol or the Narrowband IoT (NB-IoT) protocol).

In embodiments, the at least one environmental sensor can include an anemometer.

In embodiments, the emissions detector can further include at least one solar panel.

In embodiments, the emissions detector can further include an accelerometer.

In embodiments, the emissions detector can further include a camera or LIDAR device.

In embodiments, the emissions detector or parts thereof can be mounted on a pole. The pole can include a ground anchor or tripod base or other means for removably securing the pole to ground with or without the use of concrete.

In embodiments, automated systems and methods are provided for methane emissions monitoring of one or more industrial facilities. The automated systems and methods can be economically deployed worldwide on a large scale. The automated systems and methods employ a network of emissions detectors for each industrial facility to be monitored. The emissions detectors of the network are spaced from one another at different locations at the industrial facility. The emissions detectors of the network can be configured to perform time-series measurements of methane concentration at different locations within the industrial facility and communicate time-series data based on such measurements (i.e., high-frequency location-specific methane concentration data) directly to a radio access network located within communication range from the industrial facility for delivery over a data communication network to a remote cloud computing environment.

One or more emissions detectors of the network can also be configured to perform time-series measurements of atmospheric conditions (such as temperature, atmospheric pressure, and humidity) at one or more locations within the industrial facility and communicate times-series sensor data based on such measurements (i.e., high-frequency location-specific atmospheric condition data) directly to the radio access network for delivery over the data communication network to the remote cloud computing environment.

One or more emissions detectors of the network can also be configured to perform time-series measurements of environmental conditions (such as wind speed, wind direction, and solar radiation) at one or more locations within the industrial facility and communicate times-series sensor data based on such measurements (i.e., high-frequency location-specific environmental condition data) directly to the radio access network for delivery over the data communication network to the remote cloud computing environment.

Each emissions detector of the network can include a Global Navigation Satellite System (GNSS) module that precisely tracks the position of the emissions detector and time. The sensor data communicated from a given emissions detector of the network can be stamped with location and time information as recorded by the Global Navigation Satellite System (GNSS) module in synchronization with the time-series measurements performed by the given emissions detector.

The time-series data (i.e., the high-frequency location-specific methane concentration data and the high-frequency location-specific atmospheric data and the high-frequency location-specific environmental data) can be communicated from the network of emissions detectors directly to the radio access network for delivery over the data communications network to the remote cloud computing environment. The time-series data can represent i) methane concentration at specific locations within the industrial facility as a function of time as derived from the location-specific raw methane concentration data, ii) atmospheric conditions at specific location(s) within the one or more industrial facility as a function of time as derived from the location-specific atmospheric data, and iii) environmental conditions at specific location(s) within the one or more industrial facility as a function of time as derived from the location-specific environmental data.

Each emissions detector of the network can be configured to collect and process raw time-series sensor data measured by the sensors of the respective emission detector, for example, including filtering and/or averaging the raw time-series sensor data. Additionally or alternatively, the remote cloud computing environment can be configured to collect, aggregate, and process time-series data communicated from the network of emissions detectors, for example, including filtering and/or averaging the data. The remote cloud computing environment can be further configured to process the time-series data using a suitable computational model, such as Gaussian plume dispersion model, to characterize methane emission at the industrial facility. For example, the cloud computing environment can process the time-series data using a suitable computational model (e.g., Gaussian plume dispersion model) to detect the presence of methane emissions at the industrial facility, the location of the methane emissions at the industrial facility (when present), and the associated rate of methane emissions at the industrial facility (when present). The cloud computing environment can generate data related to the methane emission (such as the location of the methane emission at the industrial facility, and the associated rate of methane emission at the industrial facility) and process such data to automatically generate an alert characterizing the methane emission at the industrial facility. The alert can be communicated to a designated party or user to initiate or schedule mitigation of the methane emissions at the industrial facility.

In embodiments, each emissions detector of the network can include communication electronics that embody a RF communication modem for direct wireless data communication between the emissions detector and the radio access network. In embodiments, the wireless data communication can implement a predefined wireless communication protocol supported by both the RF communication modem and the radio access network. The predefined wireless communication protocol can have a communication range of ten kilometers or less, such as the LTE-M protocol or Narrowband IoT (NB-IoT) protocol. The wireless data communication between the emissions detector and the radio access network can be configured to communicate time-series data (i.e., the high-frequency location-specific methane concentration data and the high-frequency location-specific atmospheric data and the high-frequency location-specific environmental condition data) directly from the emissions detector to the radio access network for delivery over the data communication network to the remote cloud computing environment.

In embodiments, the network of emissions detectors can be deployed over locations within one or more oil and gas facilities, such as one or more well sites, one or more compressor stations, or one or more processing facilities.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF DRAWINGS

The subject disclosure is further described in the detailed description which follows, in reference to the noted plurality of drawings by way of non-limiting examples of the subject disclosure, in which like reference numerals represent similar parts throughout the several views of the drawings, and wherein:

FIG. 1 is a schematic block diagram that depicts an automated system for methane emissions monitoring in accordance with an embodiment of the subject disclosure;

FIG. 2 is a schematic diagram that depicts the automated system for methane emissions monitoring of FIG. 1 deployed at a well site (e.g., well pad);

FIG. 3 is a schematic diagram that depicts the automated system for methane emissions monitoring of FIG. 1 deployed at another well site (e.g., well pad);

FIG. 4 is a schematic diagram of an emissions detector with one or more environmental sensor(s) in accordance with the present disclosure, which is suitable for use as part of the system of FIG. 1; and

FIG. 5 is a schematic diagram of an example computing system.

### DETAILED DESCRIPTION

The particulars shown herein are by way of example and for purposes of illustrative discussion of the embodiments of the subject disclosure only and are presented in the cause of providing what is believed to be the most useful and readily understood description of the principles and conceptual aspects of the subject disclosure. In this regard, no attempt is made to show structural details in more detail than is necessary for the fundamental understanding of the subject disclosure, the description taken with the drawings making apparent to those skilled in the art how the several forms of the subject disclosure may be embodied in practice. Furthermore, like reference numbers and designations in the various drawings indicate like elements.

The present disclosure is directed to automated systems and methods that provide for methane emissions monitoring of one or more industrial facilities. The automated systems and methods can be economically deployed worldwide on a large scale.

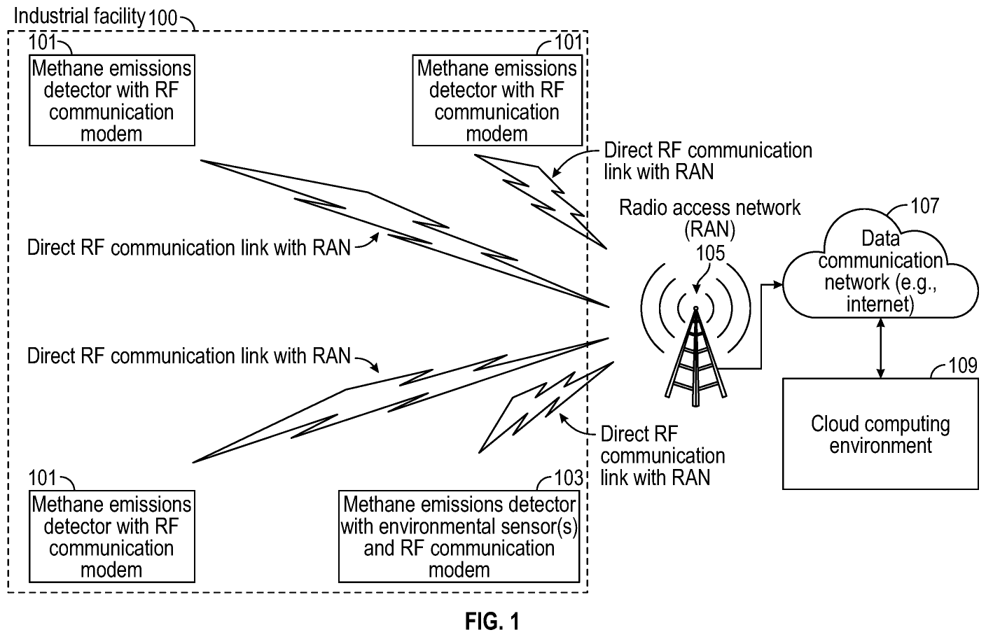

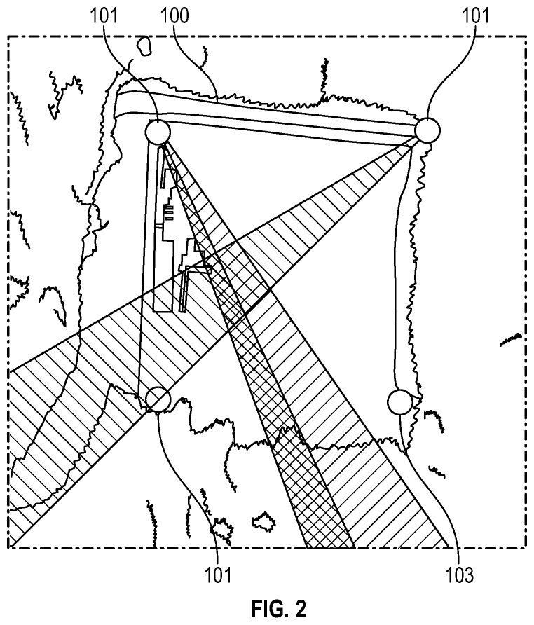

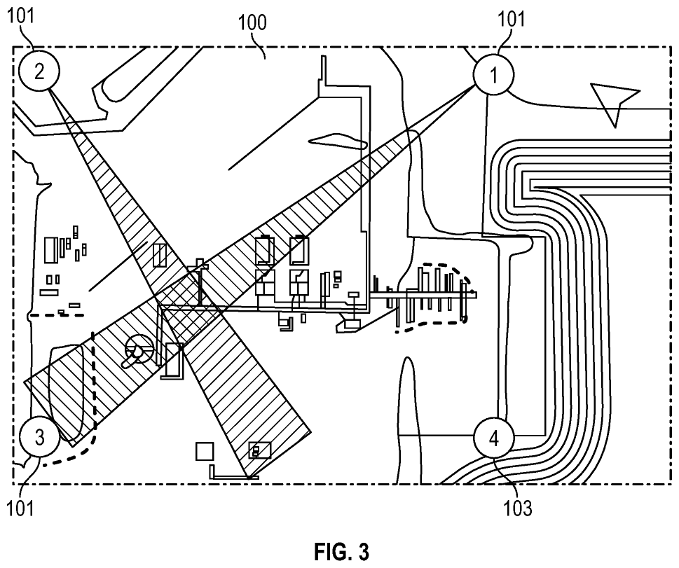

In an example embodiment shown in FIG. 1, an automated system is provided that includes a network of methane emissions detectors (for example, three methane emissions detectors 101 and one methane emissions detector with environmental sensor(s) 103) that are placed at fixed locations at or near an industrial facility (e.g., well site) 100. In embodiments, the methane emissions detectors (101, 103) of the network can be spaced from one another at different locations at the industrial facility 100, for example, in opposite corners of the facility as shown in FIGS. 2 and 3. The respective methane emissions detectors (101, 103) of the network can each include a gas sensor configurable or configured to measure concentration of methane in atmospheric gas at or near the location of the gas sensor. The respective methane emissions detectors (101, 103) of the network can also each include an RF communication modem configurable or configured to communicate times-series sensor data based on such methane concentration measurements (i.e., high-frequency location-specific methane concentration data) directly to a radio access network 105 over one or more RF communication links supported by the RF communication modem and the radio access network for delivery over the data communication network 107 to the cloud computing environment 109.

The radio access network (RAN) 105 is part of a mobile telecommunication system. Conceptually, the RAN 105 resides between wireless-enabled devices (such as the emissions detectors (101, 103) or other IOT-enabled devices) and the data communication network 107 that includes the Internet. The RAN 105 can include the following elements: antennas that convert electrical signals into radio waves, radios that transform digital information into signals that can be sent wirelessly and ensure that transmissions are in the correct frequency bands with the right power levels, and baseband units (BBUs) that provide a set of signal processing functions that make wireless communication possible. Traditional BBUs employ custom electronics combined with software to enable wireless communication. The direct RF communication links between the respective methane emissions detectors (101, 103) and the RAN 105 can provide for wireless communication of the times-series sensor data based on the methane concentration measurements from the respective methane emissions detectors (101, 103) to the RAN 105 without forwarding or communicating such data to another communication node (such as a gateway node).

In embodiments, one or more methane emissions detectors of the network can include one or more atmospheric sensors that are configurable or configured to perform time-series measurements of atmospheric conditions (such as temperature, atmospheric pressure, and humidity) at one or more locations within the industrial facility 100. The RF communication modem of the one or more methane emissions detectors with atmospheric sensor(s) can be further configurable or configured to communicate time-series data based on such measurements (i.e., high-frequency location-specific atmospheric data) directly to the RAN 105 for communication over the data communication network 107 to the cloud computing environment 109. The direct RF communication links between the respective methane emissions detectors (101, 103) and the RAN 105 can provide for wireless communication of the times-series sensor data based on the measured atmospheric conditions from one or more methane emissions detectors to the RAN 105 without forwarding or communicating such data to another communication node (such as a gateway node). In embodiments, the atmospheric sensor(s) can be selected from the group that includes: one or more sensors that measure temperature and humidity, and a barometer for measuring atmospheric pressure.

In embodiments, one or more methane emissions detectors of the network, such as the methane emissions detector 103 in FIG. 1, 2 or 3, can include one or more environmental sensors that are configurable or configured to perform time-series measurements of environmental conditions (such as wind speed, wind direction, and solar radiation) at one or more locations within the industrial facility 100. The RF communication modem of the one or more methane emissions detectors with environmental sensor(s) can be further configurable or configured to communicate time-series data based on such measurements (i.e., high-frequency location-specific environmental condition data) directly to the RAN 105 for communication over the data communication network 107 to the cloud computing environment 109. The direct RF communication links between the respective methane emissions detectors (101, 103) and the RAN 105 can provide for wireless communication of the times-series sensor data based on the measured environmental conditions from one or more methane emissions detectors to the RAN 105 without forwarding or communicating such data to another communication node (such as a gateway node). In embodiments, the environmental sensors can be selected from the group that includes: an anemometer for measuring wind speed and wind direction, and a pyranometer for measuring solar radiation.

In embodiments, each methane emissions detector (101, 103) of the network can include a Global Navigation Satellite System (GNSS) module that precisely tracks the position of the methane emissions detector and time. The time-series data communicated from a given methane emissions detector (101, 103) can be stamped with location and time information as recorded by the Global Navigation Satellite System (GNSS) module in synchronization with the time-series measurements performed by the given methane emissions detector (101, 103).

In embodiments, the time-series data can be communicated from the network of emissions detectors (101, 103) directly to the RAN 105 for delivery over the data communications network 107 to the remote cloud computing environment 109. The time-series data can represent i) methane concentration at specific locations within the industrial facility as a function of time as derived from the location-specific raw methane concentration data, ii) atmospheric conditions at specific location(s) within the one or more industrial facility as a function of time as derived from the location-specific atmospheric data, and iii) environmental conditions at specific location(s) within the one or more industrial facility as a function of time as derived from the location-specific environmental data.

Each emissions detector (101, 103) of the network can be configured to collect and process raw time-series sensor data measured by the sensors of the respective emission detector, for example, including filtering and/or averaging the raw time-series sensor data.

Additionally or alternatively, the remote cloud computing environment 109 can be configured to collect, aggregate, and process time-series data communicated from the network of emissions detectors, for example, including filtering and/or averaging the data.

In embodiments, the remote cloud computing environment 109 can be configured to process the time-series data using a suitable computational model, such as Gaussian plume dispersion model, to characterize methane emission at the industrial facility 100. For example, the cloud computing environment 109 can process the time-series data using a suitable computational model (e.g., Gaussian plume dispersion model) to detect the presence of methane emissions at the industrial facility 100, the location of the methane emissions at the industrial facility 100 (when present), and the associated rate of methane emissions at the industrial facility 100 (when present). The cloud computing environment 109 can generate data related to the methane emissions (such as the location of the methane emissions at the industrial facility 100, and the associated rate of methane emissions at the industrial facility 100) and process such data to automatically generate an alert characterizing the methane emissions at the industrial facility 100. The alert can be communicated to a designated party or user to initiate or schedule mitigation of the methane emissions at the industrial facility 100.

In embodiments, the RF communication modems of the respective emission detectors 101, 103 and the RAN 105 support wireless data communication over one or more direct RF communication links between the respective emissions detectors 101, 103 and the RAN 105. In embodiments, such direct RF communication links can implement a predefined wireless communication protocol, such as the LTE-M protocol or Narrowband IoT (NB-IoT) protocol. The LTE-M protocol and Narrowband IoT are wireless communication protocols that do not conform to the standard and licensed LTE construct. These protocols employ LPWAN (Low Power Wide Area Network) technology that operates independently in unused 200-kHz bands previously used in Global System for Mobile Communications networks known as GSM. These protocols are commonly used to connect to IIoT (Industrial IoT) devices, such as smart parking devices, wearables, utilities, and industrial solutions. These protocols employ a frequency spectrum that will not interfere with other devices, facilitating more reliable data transfer, and they can cover large areas while consuming a smaller amount of energy. Furthermore, the range of these protocols is typically limited in distance (e.g., ten kilometers or less), and thus the emission detectors 101, 103 need to be located relative to the RAN 105 within this range. The wireless data communication between the emissions detectors 101, 103 of the network and the RAN 105 can be configured to communicate time-series data from the respective emissions detectors directly to the RAN 105 for delivery over the data communication network 107 to the remote cloud computing environment 109. The direct wireless communication between the respective methane emissions detectors (101, 103) and the RAN 105 can provide for wireless communication of the times-series from the respective methane emissions detectors to the RAN 105 without forwarding or communicating such data to another communication node (such as a gateway node).

In embodiments, each methane emissions detector (101, 103) of the network can include an accelerometer for measuring the acceleration of the methane emissions detector. Detector-specific acceleration data can be communicated from the respective methane emissions detectors (101, 103) of the network directly to the RAN 105 over the direct RF communication links between the respective methane emissions detectors and the RAN 105 for delivery over the data communication network 107 to the remote cloud computing environment 109. The direct RF communication links between the respective methane emissions detectors (101, 103) and the RAN 105 can provide for wireless communication of the detector-specific acceleration from the respective methane emissions detectors to the RAN 105 without forwarding or communicating such data to another communication node (such as a gateway node). The cloud computing environment 109 can be configured to receive and process the detector-specific acceleration data to detect fall events for the respective methane emissions detectors (101, 103) of the network. The alert can be communicated to a designated party or user to initiate or schedule repair of the fallen emissions detector at industrial facility 100.

In embodiments, one or more methane emissions detectors (101, 103) of the network can further include a camera or LIDAR device.

In embodiments, the cloud computing environment 109 can be configured to process time-series data derived from the time-series data measured by the methane emissions detector networks at the multiple industrial facilities to characterize methane emission at the respective industrial facilities.

FIG. 2 depicts the automated system for methane emissions monitoring of FIG. 1 deployed at a well site (e.g., well pad).

FIG. 3 depicts the automated system for methane emissions monitoring of FIG. 1 deployed at another well site (e.g., well pad).

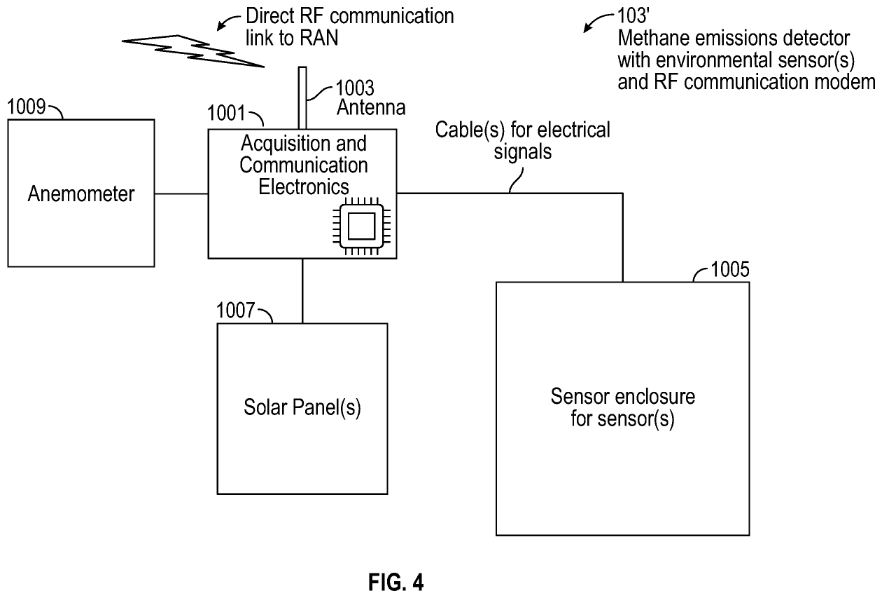

FIG. 4 is a schematic diagram of an emissions detector with one or more environmental sensor(s) 103′ in accordance with the present disclosure, which are suitable for use as part of the system of FIG. 1. The emissions detector 103′ includes acquisition and communication electronics 1001 and a sensor enclosure 1005. In embodiments, the components 1001, 1005 can be mounted to or otherwise supported by a pole (not shown) for positioning above the ground. The pole can include a ground anchor or tripod base or other means for removably securing the pole to ground with or without the use of concrete. In other embodiments, the components 1001, 1005 can be mounted to or supported by another mechanical structure above the ground.

The acquisition and communication electronics 1001 includes electrical circuitry that interfaces to electrical interface components of the sensor enclosure 1005 via one or more cable(s) as shown. The acquisition and communication electronics 1001 further includes an RF communication modem that interfaces to one or more RF antenna 103 and supports one or more direct RF communication links with a radio access network (RAN). In embodiments, the direct RF communication link(s) can employ wireless data communication according to one or more predefined wireless communication protocols (such as the LTE-M protocol or the Narrowband IoT protocol) as supported by both the RF communication modem of the acquisition and communication electronics 1001 and the RAN. The electrical circuitry of the acquisition and communication electronics 1001 can be embodied by one or more printed circuit boards (PCBs) as is well known in the electronic arts. The acquisition and communication electronics 1001 can further include or interface to an electrical power source that supplies electrical power to the electrical components of the emissions detector 103′. In embodiments, the electrical power source can include one or more pole-mounted solar panels 1107 as shown, one or more batteries (e.g., one or more lithium-ion batteries), a wind-powered generator, mains power, and/or other suitable electrical power source.

The sensor enclosure 1005 mechanically supports a gas sensor and possibly at least one atmospheric sensor. The sensor enclosure 1005 is configured to permit atmospheric gas to flow into the enclosure and into space at or near the gas sensor, and the gas sensor can be configured to measure the concentration of methane in the atmospheric gas that flows into the enclosure and into the space at or near the gas sensor. The methane can be part of the atmospheric gas that flows into the space at or near the gas sensor due to a methane leak in the local vicinity of sensor enclosure 1005. The sensor enclosure 1005 can also be configured to permit atmospheric gas to flow into the enclosure and into space at or near one or more atmospheric sensor(s), and the atmospheric sensor(s) can be configured to measure gas properties (such as temperature, atmospheric pressure, and humidity) of the atmospheric gas that flows into the enclosure and into the space at or near the atmospheric sensor(s). The gas sensor can output analog signals or digital data that represents the methane concentration measured by the gas sensor. Such analog signals or digital data can be supplied to electrical components of the sensor enclosure 1005 for processing and/or output to the acquisition and communication electronics 1001. The atmospheric sensor(s) can output analog signals or digital data that represent the atmospheric properties measured by the atmospheric sensor(s). Such analog signals or digital data can be supplied to electrical components of the enclosure 1005 for processing and/or output to the acquisition and communication electronics 1001.

The acquisition and communication electronics 1001 can further interface to an anemometer 1009, which can be configured to measure wind speed and wind direction in the local vicinity of the detector 103′.

In embodiments, the electrical components of the enclosure 1005 can provide for at least one of: sampling, analog-to-digital (A-to-D) conversion, data filtering and averaging, and/or data communication with the acquisition and communication electronics 1001 via the cable(s) therebetween. Similarly, the electrical components of the acquisition and communication electronics 1001 can provide for at least one: sampling, A-to-D conversion, data filtering and averaging, and/or data communication with the electrical components of the enclosure 1005 via the cable(s) therebetween.

The acquisition and communication electronics 1001 can be configured to generate time-series data based on the measurements made by the sensor(s) of the enclosure 1005 and the anemometer 1009 over time and communicate such time-series data directly to the RAN for delivery over a data communication network to a remote cloud computing environment for processing as described herein. Such time-series data can represent raw measurements of the sensor(s) of the enclosure 1005 and the anemometer 1009 over time. Alternatively or additionally, such time-series data can be derived from filtering and/or averaging the measurements of the sensor(s) of the enclosure 1005 and the anemometer 1009 over time. Similarly, the remote cloud computing environment 109 can be configured to filter and/or average data that represents the raw measurements of the sensor(s) of the enclosure 1005 and the anemometer 1009 over time for processing as described herein.

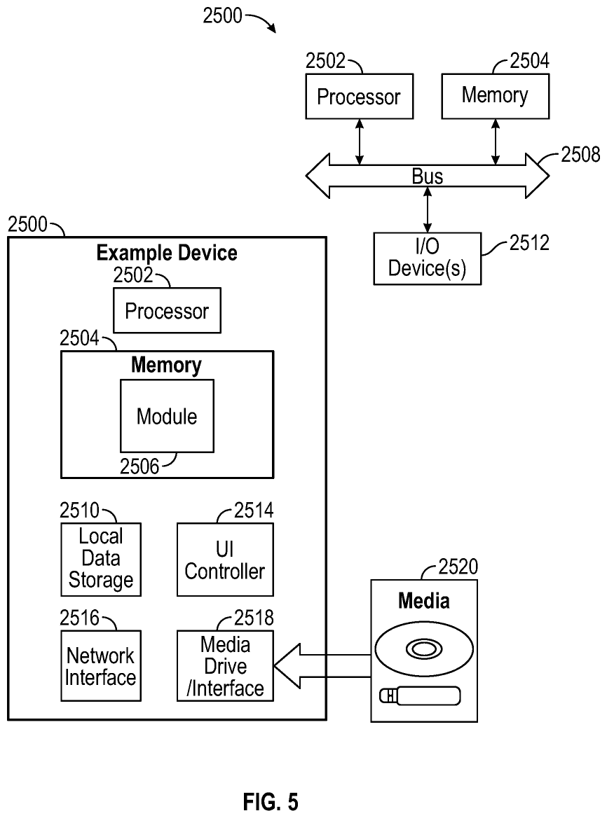

In some embodiments, the methods and system of the present disclosure may involve a computing system. FIG. 5 illustrates an example computing system 2500, with a processor 2502 and memory 2504 that can be configured to implement various embodiments of the automated systems and methods for methane emissions monitoring as discussed in the present disclosure. Memory 2504 can also host one or more databases and can include one or more forms of volatile data storage media such as random-access memory (RAM), and/or one or more forms of nonvolatile storage media (such as read-only memory (ROM), flash memory, and so forth).

Device 2500 is one example of a computing device or programmable device and is not intended to suggest any limitation as to scope of use or functionality of device 2500 and/or its possible architectures. For example, device 2500 can comprise one or more computing devices, programmable logic controllers (PLCs), etc.

Further, device 2500 should not be interpreted as having any dependency relating to one or a combination of components illustrated in device 2500. For example, device 2500 may include one or more of computers, such as a laptop computer, a desktop computer, a mainframe computer, etc., or any combination or accumulation thereof.

Device 2500 can also include a bus 2508 configured to allow various components and devices, such as processors 2502, memory 2504, and local data storage 2510, among other components, to communicate with each other.

Bus 2508 can include one or more of any of several types of bus structures, including a memory bus or memory controller, a peripheral bus, an accelerated graphics port, and a processor or local bus using any of a variety of bus architectures. Bus 2508 can also include wired and/or wireless buses.

Local data storage 2510 can include fixed media (e.g., RAM, ROM, a fixed hard drive, etc.) as well as removable media (e.g., a flash memory drive, a removable hard drive, optical disks, magnetic disks, and so forth). One or more input/output (I/O) device(s) 2512 may also communicate via a user interface (UI) controller 2514, which may connect with I/O device(s) 2512 either directly or through bus 2508.

In one possible implementation, a network interface 2516 may communicate outside of device 2500 via a connected network. A media drive/interface 2518 can accept removable tangible media 2520, such as flash drives, optical disks, removable hard drives, software products, etc. In one possible implementation, logic, computing instructions, and/or software programs comprising elements of module 2506 may reside on removable media 2520 readable by media drive/interface 2518.

In one possible embodiment, input/output device(s) 2512 can allow a user (such as a human annotator) to enter commands and information to device 2500, and also allow information to be presented to the user and/or other components or devices. Examples of input device(s) 2512 include, for example, sensors, a keyboard, a cursor control device (e.g., a mouse), a microphone, a scanner, and any other input devices known in the art. Examples of output devices include a display device (e.g., a monitor or projector), speakers, a printer, a network card, and so on.

Various systems and processes of present disclosure may be described herein in the general context of software or program modules, or the techniques and modules may be implemented in pure computing hardware. Software generally includes routines, programs, objects, components, data structures, and so forth that perform particular tasks or implement particular abstract data types. An implementation of these modules and techniques may be stored on or transmitted across some form of tangible computer-readable media. Computer-readable media can be any available data storage medium or media that is tangible and can be accessed by a computing device. Computer readable media may thus comprise computer storage media. “Computer storage media” designates tangible media, and includes volatile and non-volatile, removable, and non-removable tangible media implemented for storage of information such as computer readable instructions, data structures, program modules, or other data. Computer storage media include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible medium which can be used to store the desired information, and which can be accessed by a computer.

Some of the methods and processes described above, can be performed by a processor. The term “processor” should not be construed to limit the embodiments disclosed herein to any particular device type or system. The processor may include a computer system. The computer system may also include a computer processor (e.g., a microprocessor, microcontroller, digital signal processor, general-purpose computer, special-purpose machine, virtual machine, software container, or appliance) for executing any of the methods and processes described above.

The computer system may further include a memory such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device.

Alternatively or additionally, the processor may include discrete electronic components coupled to a printed circuit board, integrated circuitry (e.g., Application Specific Integrated Circuits (ASIC)), and/or programmable logic devices (e.g., a Field Programmable Gate Arrays (FPGA)). Any of the methods and processes described above can be implemented using such logic devices.

Some of the methods and processes described above, can be implemented as computer program logic for use with the computer processor. The computer program logic may be embodied in various forms, including a source code form or a computer executable form. Source code may include a series of computer program instructions in a variety of programming languages (e.g., an object code, an assembly language, or a high-level language such as C, C++, or JAVA). Such computer instructions can be stored in a non-transitory computer readable medium (e.g., memory) and executed by the computer processor. The computer instructions may be distributed in any form as a removable storage medium with accompanying printed or electronic documentation (e.g., shrink wrapped software), preloaded with a computer system (e.g., on system ROM or fixed disk), or distributed from a server or electronic bulletin board over a communication system (e.g., the Internet or World Wide Web).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words ‘means for’ together with an associated function.

What is claimed is:

1. An emissions detector for monitoring methane emissions at one or more industrial facilities, the emissions detector comprising:
a gas sensor that is configurable or configured to measure concentration of methane in atmospheric gas; and
an RF communication modem, operably coupled to the gas sensor, that is configurable or configured for direct RF communication with a radio access network.

2. An emissions detector according to claim 1, wherein:
the RF communication modem is configurable or configured to wirelessly communicate time-series data based on methane concentration measurements of the gas sensor directly to the radio access network for delivery to a cloud computing environment.

3. An emissions detector according to claim 2, wherein:
the RF communication modem and the radio access network are configured to support wireless data communication of the time-series data over at least one direct RF communication link between the RF communication modem and the radio access network, wherein the at least one direct RF communication link implements at least one predefined wireless communication protocol having a range of ten kilometers or less (preferably the LTE-M protocol or the Narrowband IoT (NB-IoT) protocol).

4. An emissions detector according to claim 1, further comprising:
at least one atmospheric sensor that is configurable or configured to measure properties of atmospheric gas, wherein the RF communication modem is operably coupled to the at least one atmospheric sensor.

5. An emissions detector according to claim 4, wherein:
the RF communication modem is configurable or configured to wirelessly communicate time-series data based on measurements of the at least one atmospheric sensor directly to the radio access network for delivery to the cloud computing environment.

6. An emissions detector according to claim 5, wherein:
the RF communication modem and the radio access network are configured to support wireless data communication of the time-series data over at least one direct RF communication link between the RF communication modem and the radio access network, wherein the at least one direct RF communication link implements at least one predefined wireless communication protocol having a range of ten kilometers or less (preferably the LTE-M protocol or the Narrowband IoT (NB-IoT) protocol).

7. An emissions detector according to claim 4, wherein:
the properties of atmospheric gas measured by the at least one atmospheric sensor are selected from the group including: temperature, atmospheric pressure, and humidity.

8. An emissions detector according to claim 1, further comprising:
at least one environmental sensor that is configurable or configured to measure environmental conditions, wherein the RF communication modem is operably coupled to the at least one environmental sensor.

9. An emissions detector according to claim 8, wherein:
the RF communication modem is configurable or configured to wirelessly communicate time-series data based on measurements of the at least one environmental sensor directly to the radio access network for delivery to the cloud computing environment.

10. An emissions detector according to claim 9, wherein:
the RF communication modem and the radio access network are configured to support wireless data communication of the time-series data over at least one direct RF communication link between the RF communication modem and the radio access network, wherein the at least one direct RF communication link implements at least one predefined wireless communication protocol having a range of ten kilometers or less (preferably the LTE-M protocol or the Narrowband IoT (NB-IoT) protocol).

11. An emissions detector according to claim 8, wherein:
the environmental conditions measured by the at least one environmental sensor are selected from the group including: wind speed, wind direction, and solar radiation.

12. An emissions detector according to claim 1, wherein:
the RF communication modem is part of acquisition and communication electronics of the emissions detector.

13. An emissions detector according to claim 1, further comprising at least of:
at least one solar panel;
an anemometer;
an accelerometer; or
a camera or LIDAR device.

14. A system for monitoring methane emissions at one or more industrial facilities, the system comprising:
a network of emissions detectors spaced from one another at different locations within an industrial facility, wherein each emissions detector of the network includes a gas sensor that is configurable or configured to measure concentration of methane in atmospheric gas, and an RF communication modem, operably coupled to the gas sensor, that is configurable or configured for direct RF communication with a radio access network; and
a cloud computing environment operably coupled to the network of emissions detectors via the radio access network;
wherein the network of emissions detectors is configured to perform time-series measurements at different locations within the industrial facility and wirelessly communicate time-series data based on such measurements directly to the radio access network for delivery to the cloud computing environment; and
wherein the cloud computing environment is configured to receive and process the time-series data to detect and characterize methane emission at the industrial facility.

15. A system according to claim 14, wherein:
the cloud computing environment is configured to process the time-series data in conjunction with a computational model to determine the location of the methane emission at the industrial facility and the associated rate of methane emission at the industrial facility.

16. A system according to claim 15, wherein:
the computational model comprises a Gaussian plume dispersion model.

17. A system according to claim 14, wherein:
the time-series data represents methane concentration at specific locations within the industrial facility and environmental conditions at specific location(s) within the industrial facility as a function of time; and
wherein the cloud computing environment is configured to process such time-series data in conjunction with a computation model that simulates methane emission at the industrial facility based on environmental conditions within the industrial facility.

18. A system according to claim 14, wherein:
the cloud computing environment is further configured to generate data related to the methane emission and process such data to automatically generate an alert characterizing the methane emission at the industrial facility.

19. A system according to claim 14, wherein:
the industrial facility comprises an oil and gas facility such as a well site, compressor station, or processing facility.

20. A system according to claim 14, wherein:
the RF communication modems of the respective emissions detectors of the network and the radio access network are each configured to support wireless data communication of the time-series data over at least one direct RF communication link between the RF communication modem and the radio access network, wherein the at least one direct RF communication link implements at least one predefined wireless communication protocol having a range of ten kilometers or less (preferably the LTE-M protocol or the Narrowband IoT (NB-IoT) protocol).
